(19) United States
(12) Patent Application Publication
(10) Pub. No.: US 2025/0213384 A1
(43) Pub. Date: Jul. 3, 2025

(54) COUPLING SYSTEM FOR OSTOMY APPLIANCES

(71) Applicant: ConvaTec Inc., Bridgewater, NJ (US)

(72) Inventor: James Jones, Stokesdale, NC (US)

(21) Appl. No.: 19/006,787

(22) Filed: Dec. 31, 2024

Related U.S. Application Data
(63) Continuation of application No. PCT/US23/69267, filed on Jun. 28, 2023.
(60) Provisional application No. 63/357,851, filed on Jul. 1, 2022.

(51) Int. Cl. A61F 5/443; A61F 5/448

(52) U.S. Cl. CPC A61F 5/443; A61F 5/448; A61F 2005/4486

(57) ABSTRACT

The present disclosure is a coupler assembly for an ostomy appliance. The coupler assembly has a body side coupler defining a female receiver and configured to be selectably coupled to a user and a bag side coupler defining a male portion sized to be selectively coupled to the female receiver of the body side coupler, the bag side coupler configured to be coupled to an ostomy bag. When the male portion is coupled to the female receiver providing, a pulling force on the ostomy bag increases a decoupling force required to uncouple the male portion from the female receiver relative to when the male portion is coupled to the female receiver in a neutral state.

### CROSS-REFERENCE TO RELATED DISCLOSURES

The present disclosure claims is a continuation of International Application No. PCT/US23/69267 filed Jun. 28, 2023 and claims the benefit of U.S. Provisional Application No. 63/357,851 filed on Jul. 1, 2022, the contents of which are hereby incorporated herein in entirety.

### FIELD OF THE DISCLOSURE

The present invention relates to an ostomy coupling for removably attaching an ostomy appliance to a body fitment worn on the body of an ostomate and more specifically to providing a body fitment with a coupling part providing enhanced coupling.

### BACKGROUND OF THE DISCLOSURE

The present disclosure relates to devices that attach to a patient's body after they have undergone a surgical ostomy procedure to address a problem with their gastrointestinal tract. The varied problems they experience interfere with their body's normal processes for processing and collecting the waste products from the digestive processes. Ostomates, as these patients are referred to, are typically required to wear and maintain these waste-collection devices for the remainder of their life. At their essence, the devices are typically comprised of an adhesive “doughnut” surrounding the stoma's exit from their abdomen. The adhesive adheres to the patient on one side and attaches to a collection bag on the other. The system is intended during operation to provide a liquid and reasonably gas-tight seal so the ostomate can enjoy an active and socially engaged life.

U.S. Pat. Nos. 4,419,100, 4,610,676, 4,610,677, 5,730,735, 5,312,382, 8,366,695 (hereinafter “the '695 Patent”), and U.S. Pat. No. 8,382,732 (hereinafter “the '732 Patent”) teach an ostomy coupling designed to reduce discomfort that some ostomates experience as a result of an attachment force applied through the body fitment to the skin, when two coupling parts are pressed together. The problem of discomfort is especially important during post-operative care, when the stoma surgery is recent and the abdominal skin is very tender. These documents teach a flexible collar suspension permanently secured between the adhesive wafer and the coupling part of the body fitment. The flexible collar allows the coupling part to be displaced away from or “float” with respect to the adhesive wafer, at least by a sufficient amount for a user to insert one or more fingers behind the coupling part, in order to manually support the coupling part against the attachment force when the appliance is pressed against the body fitment. Such a coupling part is generally referred to in the art as being a floating coupling part.

Frequently, these ostomy devices are designed so that the collection bag can be detached from the adhesive via coupling flanges. This allows a fresh bag to be reattached without disturbing the underlying adhesive doughnut. This extends useable life of the adhesive, reducing cost, and minimizes skin irritation to the patient from more frequent adhesive changes. Commonly, products with this configuration are referred to as “two-piece” ostomy devices.

Another drawback to the typical designs is that the forces required to decouple and recouple the flanges are reacted directly back against the patient's abdomen. The '695 Patent and the '732 Patent teach two-piece ostomy devices designed to reduce the discomfort that many ostomates experience as a result of the attachment and detachment forces applied through the coupling.

The '695 design focuses on the period immediately following initial operative care when the wound is particularly swollen and sensitive. Doctors and post-operative staff are attending to the patient during this time. The coupling is fixed an extended distance away from the patient. This provides space for attending staff to easily support the coupling during detachment and reattachments, thereby isolating forces from the patient's abdomen.

The '732 Patent addresses the conditions found during the more common, normal, daily routine the patient experiences while wearing the collection appliance after the operative recovery period is over. The coupling can temporarily extend (or “float”) via the flexible membrane. This improves access for maintenance care by allowing the space for the wearer to insert one or more fingers behind the coupling. The wearer can support and isolate the reaction forces from decoupling and recoupling the flanges. The coupling can then collapse back to a flat position for wearing, improving comfort and appearance under clothing. The '732 Patent describes a preferred embodiment where the coupling is in fact “bistable” (i.e. holding stable in the collapsed state to resist movement or sagging for active wearers and holding stable in the extended state while the wearer is manipulating the bag coupling).

In the typical two-piece ostomy device the body-side and bag-side coupling flanges are manufactured separately from the flexible membrane. The flexible membrane is then subsequently coupled to the body-side flange and bag-side flange. In typical two-piece device, the attachment of the flexible membrane to the body-side flange has proven problematic. More specifically, the extensible membrane is typically produced by first extruding a thin, flat, flexible film made from a polyethylene copolymer or the like. The film is rolled and subsequently fed into a vacuum thermoforming operation to achieve a folded “accordion” shape. The accordion shapes are then die-cut into individual components with the required inner and outer diameter dimensions. The copolymer used for extrusion is selected to optimize flexibility while still providing a stiff-enough structure to achieve bi-stability.

The thin-film extrusion is prone to potential inclusion of small air bubbles and foreign particulate, resulting in pinholes through the film. This requires careful process control. The thermoforming process is inherently unable to control wall thickness, resulting in particularly thin walls where the film is required to stretch the most. These two factors combine to give rise to intermittently occurring small holes through the membrane that are only detectable after a subassembly has been produced and leak tested.

The body-side flange and the accordion membrane are brought together and joined with a heat-welding operation to create a subassembly for further processing into finished product. Handling the thin membrane with automated assembly equipment and placing the membrane precisely concentric with the flange prior to welding has proven challenging. The subsequent welding operation and in-process leak and dimensional testing have all combined to be the source of significant, non-recoverable scrap.

The present disclosure addresses these issues, among others, that are problematic in the prior art.

### SUMMARY

The present disclosure is a coupler assembly for an ostomy appliance. The coupler assembly has a body side coupler defining a female receiver and configured to be selectably coupled to a user and a bag side coupler defining a male portion sized to be selectively coupled to the female receiver of the body side coupler, the bag side coupler configured to be coupled to an ostomy bag. When the male portion is coupled to the female receiver providing, a pulling force on the ostomy bag increases a decoupling force required to uncouple the male portion from the female receiver relative to when the male portion is coupled to the female receiver in a neutral state.

In one example of this embodiment, the female receiver defines a locking rim to selectively couple the male portion thereto. As part of this example, the male portion defines a locking shelf, the locking shelf positioned to correspond with the locking rim of the female receiver in a coupled configuration. In another aspect of this configuration, the ostomy bag is coupled to the bag side coupler at a location radially outward of the locking shelf relative to a coupler through-hole. In another aspect of this embodiment the body side coupler is coupled to a bridging ring at a location radially inward from the locking rim relative to the coupler through-hole such that when the pulling force is applied to the ostomy bag the locking rim and the locking shelf are at least partially forced towards one another relative to the neutral state.

Another example of this embodiment has an accordion membrane defined in the body side coupler between the female receiver and a bridging ring weld. In this example, the female receiver and the accordion membrane are formed of the same material in the same manufacturing process.

Another example of this embodiment has a deflectable seal defined about the male portion at a radially inward location from the coupler through-hole relative to the locking shelf. Part of this example has a seal wall defined in the female receiver wherein the deflectable seal contacts the seal wall to substantially seal an interior of the ostomy bag from the surrounding environment when the male portion is coupled to the female receiver.

Yet another example of this embodiment has a bridging ring coupled to the body side coupler on a first side and having an adhesive on a second side, the adhesive configured to be selectively coupled to a user's abdominal wall about a stoma.

In another example of this embodiment, the body side coupler is formed of polyethylene. In one part of this example, the bag side coupler is formed of EVA.

In another example of this embodiment the female receiver of the body side coupler is welded directly to a wafer. Yet another example of this embodiment has a flange defined by the bag side coupler extending radially outward relative to a coupler through-hole, the ostomy bag being welded to the bag side coupler along a surface of the flange. In yet another example of this embodiment, the female receiver of the body side coupler has a convex flange that is offset from a planar surface of the female receiver.

Yet another embodiment of the present disclosure is a method of manufacturing a coupler assembly for an ostomy appliance. The method includes forming a body side coupler that defines a female receiver, forming a bag side coupler that defines a male portion sized to be selectively coupled to the female receiver, coupling the body side coupler to a bridging ring, and coupling the bag side coupler to an ostomy bag. In this embodiment, the male portion is coupleable to the female receiver in a coupled configuration wherein providing a pulling force on the ostomy bag increases a decoupling force required to uncouple the male portion from the female receiver relative to when the male portion is coupled to the female receiver in a neutral state.

Part of this embodiment includes forming an accordion membrane in the body side coupler between the female receiver and the bridging ring. One aspect of this example includes forming the female receiver and the accordion membrane in the same manufacturing step. In another aspect of this example, the coupling the body side coupler to the bridging ring step comprises welding a portion of the accordion membrane to the bridging ring along a bridging ring weld.

Another example of this embodiment includes coupling an adhesive to the bridging ring to selectively couple the body side coupler to a user's abdominal wall. Yet another example includes welding the ostomy bag to a flange formed in the bag side coupler along a bag weld.

Yet another example of this embodiment includes forming a locking rim in the female receiver and forming a locking shelf in the male portion, wherein the locking rim and locking shelf correspond with one another to substantially maintain the body side coupler and the bag side coupler in the coupled configuration. Part of this example includes forming a deflectable seal in the male portion and a seal wall in the female receiver, wherein the deflectable seal provides a fluid seal between the bag side coupler and the body side coupler when in the coupled configuration. In one aspect of this part, the deflectable seal is positioned to provide a biasing force of the locking shelf towards the locking rim to maintain the coupled configuration.

In yet another example of this embodiment the body side coupler and bag side coupler are formed from an injection molding process.

### BRIEF DESCRIPTION OF THE DRAWINGS

The above-mentioned aspects of the present disclosure and the manner of obtaining them will become more apparent and the disclosure itself will be better understood by reference to the following description of the embodiments of the disclosure, taken in conjunction with the accompanying drawings, wherein:

FIG. 1 is an elevated perspective view of a coupler assembly;

FIG. 2 is an elevated perspective view of a body side coupler from the coupler assembly of FIG. 1;

FIG. 3 is an elevated perspective view of a bag side coupler from the coupler assembly of FIG. 1;

FIG. 4 is a side view of the bag side coupler if FIG. 3;

FIG. 5 is an elevated perspective cross-section view of the coupler assembly of FIG. 1 coupled to an abdominal wall about a stoma;

FIG. 6 is a side section view of FIG. 5;

FIG. 7 is a detailed side section view of FIG. 5;

FIG. 8 is a detailed section side view of the bag side coupler of FIG. 3;

FIG. 9 is a detailed section side view of the body side coupler of FIG. 2;

FIG. 10 is a detailed section side view of another embodiment of a body side coupler;

FIG. 11 is a detailed section side view of another embodiment of a body side coupler assembly; and

FIG. 12 is an exemplary manufacturing flow chart for a coupler assembly.

Corresponding reference numerals are used to indicate corresponding parts throughout the several views.

### DETAILED DESCRIPTION

The embodiments of the present disclosure described below are not intended to be exhaustive or to limit the disclosure to the precise forms in the following detailed description. Rather, the embodiments are chosen and described so that others skilled in the art may appreciate and understand the principles and practices of the present disclosure.

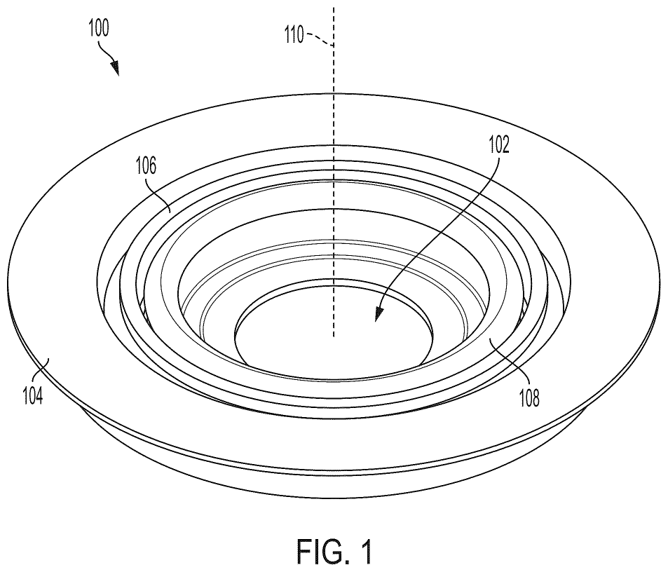

The present disclosure relates to a coupler assembly 100 for an ostomy device as illustrated in FIG. 1. The coupler assembly 100 is configured to define a coupler through-hole 102 defined about an axis 110 and sized to be positioned around a stoma of a user or “ostomate.” The coupler assembly 100 has a bag side coupler 104 selectively coupled to a body side coupler 106. The bag side coupler 104 is typically coupled to a bag or the like and the body side coupler 106 is typically coupled to the ostomate about the stoma. When the bag side coupler 104 is coupled to the body side coupler 106 and positioned about the stoma of the ostomate, material from the ostomate may pass through the coupler through-hole 102 and enter and be retained within the bag for further processing. When the bag is full of material, the bag side coupler 104 may be removed from the body side coupler 106 so the bag and material therein may be disposed, cleaned, or the like. Subsequently, a clean bag having a bag side coupler 104 may be coupled to the body side coupler 106 to capture material produced through the stoma.

The coupler assembly 100 may have an accordion membrane 108 defined in the body side coupler 106. The accordion membrane 108 may allow the body side coupler 106 to become partially spaced from the stoma of the ostomate without requiring the body side coupler 106 to become uncoupled from the ostomate. More specifically, the accordion membrane 108 may have one or more fold defined therein that allow the body side membrane 106 to be positioned adjacent to the ostomate's skin around the stoma in a first orientation, and become spaced from the ostomate's skin in a second, extended orientation. The accordion membrane allows the coupler assembly 100 to be spaced from the ostomate's skin while remaining coupled to the ostomate to further allow easy coupling and uncoupling of the bag side coupler 104 to the body side coupler 106.

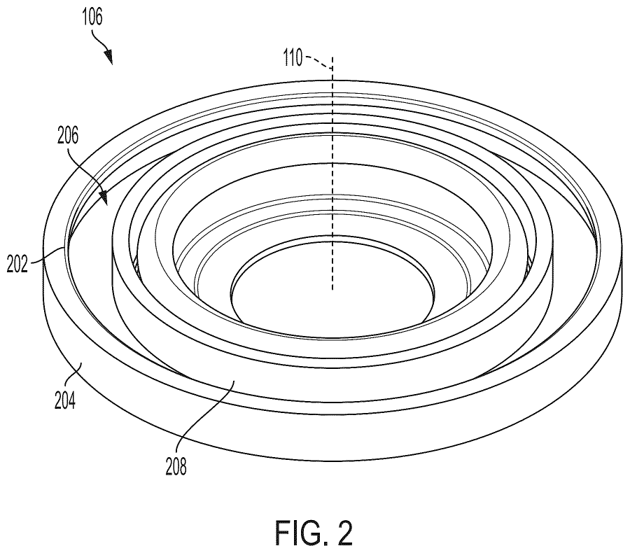

Referring now to FIG. 2, the body side coupler 106 is illustrated separated from the bag side coupler 104. The body side coupler 106 has a locking rim 202 extending radially inward towards the axis 110 from an outer wall 204. The body side coupler 106 generally defines a female receiver 206 about the axis 110. The female receiver 206 is an annular cavity within which a portion of the bag side coupler 104 may be positioned. Further, the locking rim 202 of the body side coupler 106 may interact with a locking shelf of the bag side coupler 104 to substantially prevent the bag side coupler 104 from unintentionally uncoupling from the body side coupler 106 as will be described in more detail herein.

A seal wall 208 may also be defined in the body side coupler 106 to partially form a radially inner portion of the female receiver 206 relative to the outer wall 204. The seal wall 208 may provide a sealing surface for a corresponding deflectable seal of the bag side coupler 104 to thereby substantially seal the perimeter of the female receiver 206 to a corresponding male portion of the bag side coupler 104. In the coupled configuration, the bag side coupler 104 is coupled to the body side coupler 106 such that fluid cannot substantially pass between the bag side coupler 104 and the body side coupler 106 at the female receiver 206. In other words, the stoma of the ostomate and interior cavity of the bag are substantially fluidly isolated from the surrounding environment when the bag side coupler 104 is properly coupled to the body side coupler 106.

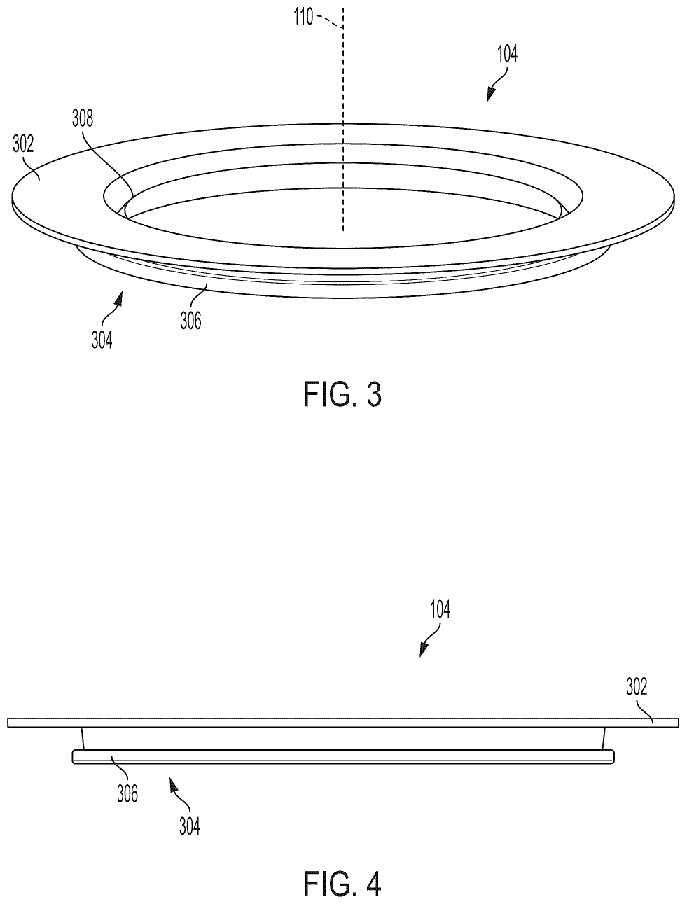

Referring now to FIGS. 3 and 4, the bag side coupler 104 is illustrated isolated from the body side coupler 106 and the corresponding bag. The bag side coupler 104 defines a radially extending flange 302 configured to provide a coupling surface for the bag. The flange 302 may be coupled to the bag through any known coupling method, such as adhesives, ultrasonic welding, or any other known coupling technique. The flange 302 may extend radially away from a male portion 304 of the bag side coupler 104. The male portion 304 is sized to be selectively positioned within the female receiver 206 of the body side coupler 106. The male portion 304 has a locking shelf 306 defined in the male portion 304 about the axis 110. The locking shelf 304 is a radial extension of the male portion 304 away from the axis 110 to define a locking interface between the locking shelf 306 and the locking rim 202 when the bag side coupler 104 is coupled to the body side coupler 106.

The male portion 304 of the bag side coupler 104 may also define a deflectable seal 308 configured to be partially positioned within the female receiver 206. The deflectable seal 308 may be sized to elastically deform radially away from the axis 110 through contact with the seal wall 208 as the bag side coupler 104 is coupled to the body side coupler 106. Once the male portion 304 of the bag side coupler 104 is fully positioned within the female receiver 206 of the body side coupler 106, the deflectable seal 308 forms a fluid seal against the seal wall 208 thereby preventing fluid from passing from the interior of the bag to the surrounding environment through the coupler assembly 100 when the coupler assembly 100 is properly positioned around the stoma of the ostomate.

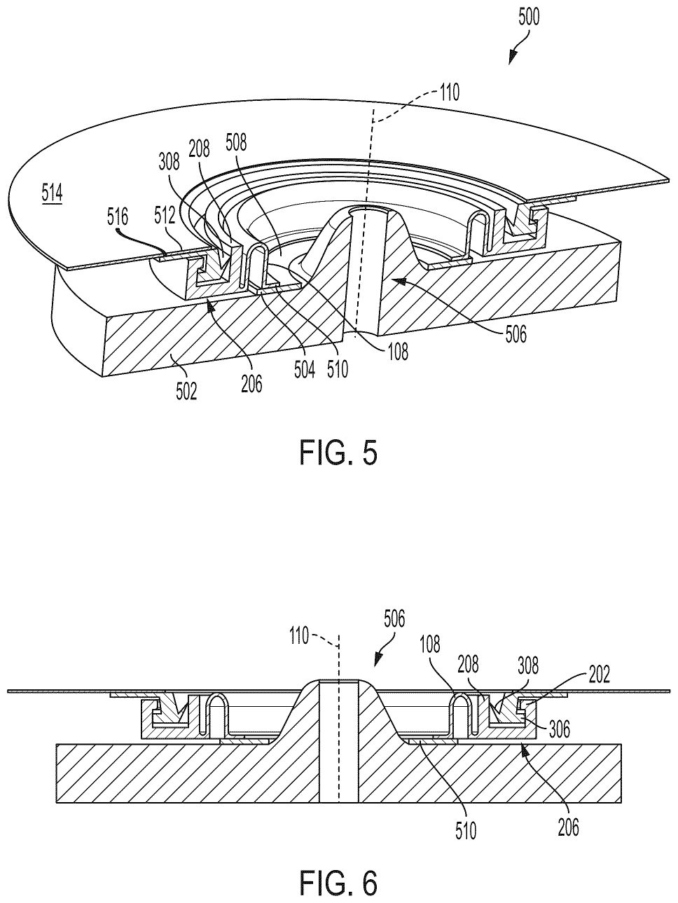

Referring now to FIGS. 5 and 6, a section view of the coupler assembly 100 is illustrated coupled to an ostomate. More specifically, the body side coupler 106 is coupled to an abdominal wall 502 of an ostomate. The body side coupler 106 is coupled to the abdominal wall 502 through an adhesive 504. As mentioned herein, the accordion member 108 may separate the adhesive portion of the body side coupler 106 from the female receiver 206 such that the female receiver 206 may become spaced from the abdominal wall 502 as the accordion member 108 unfolds to provide ease of access to the coupler assembly 100 without affecting the adhesive 504. The coupler assembly 100 may be coupled to the abdominal wall 502 with the adhesive 504 about a stoma 506 of the ostomate.

The adhesive 504 may be coupled to a body-side surface of a bridging ring 508. The bridging ring 508 may further be coupled to a portion of the accordion membrane on an opposite side relative to the body-side surface of the bridging ring 508. The bridging ring 508 may be coupled to the accordion membrane 108 using any known coupling method. For example, a bridging ring weld 510 may couple the bridging ring 508 to the accordion membrane 108 utilizing ultrasonic welding techniques. Alternatively, adhesives may be used to couple the adjacent surfaces of the bridging ring 508 and the accordion membrane 108 to one another.

While a bridging ring 508 is illustrated as a separate component from the accordion membrane 108, in one embodiment considered herein the bridging ring 508 and the accordion membrane 108 may be formed as the same component. That is to say, the bridging ring 508 is formed during the same manufacturing process as the remaining components of the body side coupler 106.

FIGS. 5 and 6 also more clearly illustrate the deflectable seal 308 positioned in a coupled configuration 500 against the seal wall 208. As discussed herein, as the bag side coupler 104 is coupled to the body side coupler 106, the deflectable seal 308 may deflect radially away from the axis 110 through contact with the seal wall 208. More specifically, as the bag side coupler 104 is moved towards the coupled configuration 500 as illustrated in FIG. 5, the locking shelf 306 of the bag side coupler 104 bypasses the locking rim 202 of the body side coupled to allow the male portion 304 of the bag side coupler 104 to be positioned within the female receiver 206 of the body side coupler 106. In the coupled configuration 500, the deflectable seal 308 is positioned at least partially pressing against the seal wall 208 about the entire perimeter around the seal wall 208. In this configuration, a substantially fluid-tight seal is formed between the bag side coupler 104 and the body side coupler 106 around the seal wall 208.

As mentioned herein, the bag side coupler 104 may provide a flange 512 that extends radially away from the male portion 304 relative to the axis 110. The flange 512 provides a surface to which a bag 514 may be coupled. The bag 514 may provide a substantially contained inner region wherein when the bag 514 is coupled to the flange 512 and the bag side coupler 104 is coupled to the body side coupler 106 and the assembly 100 is coupled to an ostomate about the stoma 506, fluid such as effluent passed through the stoma 506 is captured within the inner region of the bag 514. The bag 514 may be coupled to the flange 512 using any known technique. In one example, the bag 514 is coupled to the flange 512 through an ultrasonic welding technique that creates a bag weld 516. Alternatively an adhesive may be used to couple the bag 514 to the flange 512.

In the coupled configuration 500, the male portion 304 is position within the female receiver 206 such that the locking shelf 306 of the male portion 304 is positioned axially inside of the locking rim 202 within the female receiver 206. The outer diameter of the locking shelf 306 may be slightly larger than the inner diameter of the locking rim 202 such that transitioning the male portion 304 into the female receiver 206 requires some deflection of one or both of the locking shelf 306 and the locking rim 202. Once in the coupled configuration 500, the locking shelf 306 and locking rim 202 may return to their undeflected position. Once the male portion 304 is properly seated in the female receiver 206, the positioning of the locking shelf 306 within the female receiver 206 next to the locking rim 202 may substantially prevent the bag side coupler 104 from unintentionally separating from the body side coupler 106. More specifically, if the bag side coupler 104 is pulled axially away from the body side coupler 106 along the axis 110, the locking shelf 306 will contact the locking rim 202 to substantially prevent unintentional decoupling of the bag side coupler 104 from the body side coupler 106.

The corresponding dimensions of the locking rim 202 and locking shelf 306 may be selected so the user can easily couple the bag side coupler 104 to the body side coupler 106. However, once in the coupler configuration 500, the locking rim 202 and locking shelf 306 may substantially prevent the assembly 100 from unintentionally uncoupling under normal use conditions. That is to say, the contact between the locking rim and the locking shelf 306 may prevent the assembly 100 from uncoupling under normal weight and movement of the bag 514 and any contents therein. However, the bag side coupler 104 and the body side coupler 106 may be separated from one another when specifically manipulated by the ostomate to pull the male portion 304 from the female receiver 206. Accordingly, this disclosure contemplates utilizing different dimensions of the locking rim 202 and locking shelf 306 to provide different coupling and uncoupling force requirements. For example, if the radial overlap of the locking shelf 306 and locking rim 202 is increased, the required coupling and uncoupling force to transition the bag side coupler 104 and body side coupler 106 between the coupled configuration 500 and the separated configuration may be greater than a relatively smaller radial overlap. Accordingly, this disclosure contemplates selecting the dimensions for the locking rim 202 and locking shelf 306 that allow a user to easily couple and uncouple the assembly 100 by manipulating the bag side coupler 104 and body side coupler 106 with their hands. However, the assembly 100 the dimensions are also configured to prevent the bag side coupler 104 from separating from the body side coupler 106 under normal use conditions, such as the bag 514 being filled with effluent.

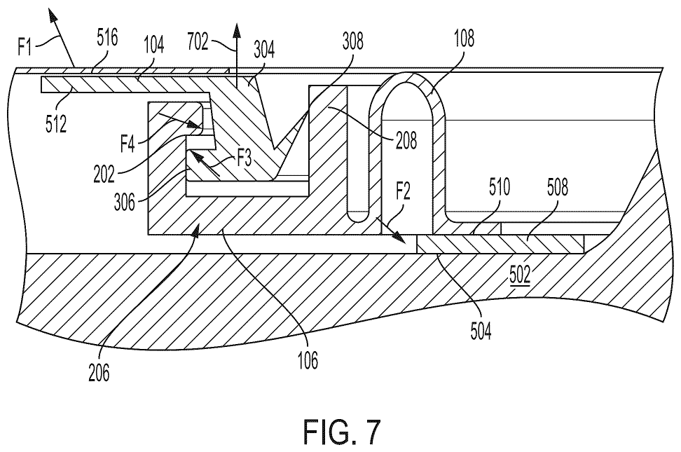

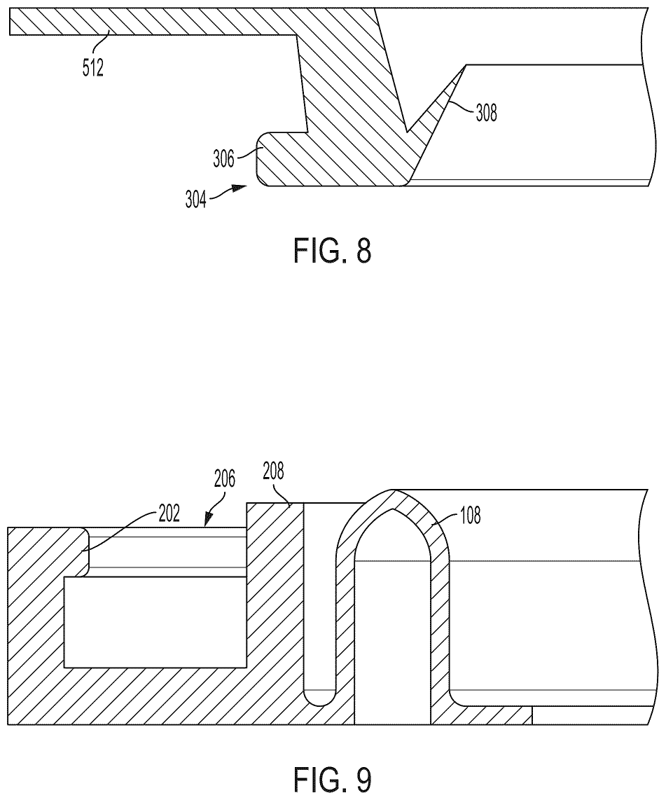

Referring now to FIG. 7, a detailed section view of the male portion bag side coupler 104 coupled to the body side coupler 106 is illustrated along with illustrative forces F1-F4 that may be expected during use. More specifically, pulling force F1 may be applied to the bag side coupler 104 by the bag 514 as it is pulled away from the user. The pulling force F1 may be applied by the bag 514 to the bag side coupler at the bag weld 516. As one example, the pulling force F1 may be the expected force applied to the bag side coupler 104 when the bag 514 is full of effluent. Alternatively, the pulling force F1 may be caused by the bag 514 being inadvertently pulled by the user or the like. Regardless, bag side coupler 104 and body side coupler 106 may be permitted to extend slightly away from the ostomate responsive to the force F1 by unfolding the accordion membrane 108.

However, once the accordion membrane 108 is fully extended, if the pulling force F1 persists, a responsive force F2 will occur where the accordion membrane 108 attaches to the female receiver 206. The responsive force F2 may substantially counter the pulling force F1 when the accordion membrane 108 cannot unfold any further. In this configurations, the pulling force F1 is passed from the bag 514, through the male portion 304 and female receiver 206, through the accordion membrane 108 to the bridging ring 508 through the bridging ring weld 510, and to the abdominal wall 502 via the adhesive 504. If the pulling force F1 sufficiently exceeds a decoupling force 702 (i.e., the force required to uncouple the bag side coupler 104 from the body side coupler 106), the bag side coupler 104 may be separated from the body side coupler 106 as the male portion 304 exits the female receiver 206.

The pulling force F1 and responsive force F2 also cause a shelf force F3 and a rim force F4 where the locking shelf 306 and locking rim 202 are adjacent to one another. The shelf force F3 may be directed at least partially towards the locking rim 202 because of the radially offset location where the bag 514 is coupled to the flange 512. In other words, because the pulling force F1 is being applied to a location radially away from the locking shelf 306, the shelf force F3 applied to resist the pulling force F1 may drive the locking shelf 306 towards the locking rim 202. Similarly, the rim force F4 may be directed radially inward towards the locking shelf 306. This may occur because the locking rim 202 is radially away from the location of the responsive force F2 acting on the female receiver 206. As such, the responsive force F2 may cause the rim force F4 to be oriented towards the locking shelf 306.

By coupling the bag 514 to the bag side coupler 104 at a location radially away from the locking shelf 306, when the pulling force F1 is applied the male portion 304 is going to be biased into the locking rim 202 because the locking shelf 306 will tend to slightly pivot into the locking rim 202 due to the radially outward pulling force. Similarly, by coupling the body side coupler 106 to the ostomate at a radially inner portion of the body side coupler 106 relative to the locking rim 202, when the responsive force F2 resists the pulling force F1 the female receiver 206 is going to be biased to pivot the locking rim 202 into the locking shelf 306. This configuration creates an increased required decoupling force 702 when the bag 514 is pulling the bag side coupler 104 from the body side coupler 106 compared to when the user is separating the bag side coupler 104 from the body side coupler 106 as described herein.

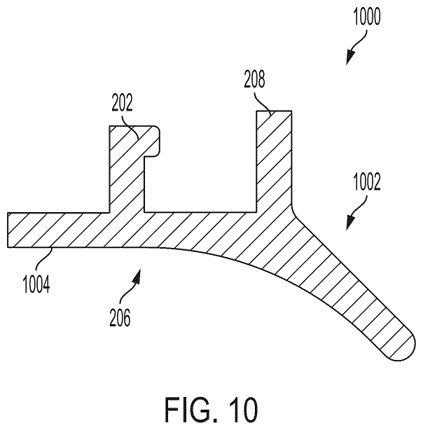

The above-described forces allow the bag side coupler 104 and body side coupler 106 to substantially resist becoming uncoupled from one another when a pulling force is applied to the bag side coupler 104. This may prevent the coupler assembly from becoming unintentionally uncoupled due to the weight or other movement acting on the bag. Rather, to uncouple the bag side coupler 104 from the body side coupler 106, the user may directly manipulate the bag side coupler 104 and the body side coupler 106 with their hands. Further, the accordion membrane 108 allows the user to move the body side coupler 106 slightly away from the abdominal wall 502 to allow for access to the body side coupler 106. In one aspect of this disclosure, the body side coupler 106 may have a tab (similar to tab 1004 from FIG. 10) extending radially from the female receiver 206. In use, the user may manipulate the tab with one hand and the flange 512 of the bag side coupler 104 to uncouple the bag side coupler 104 from the body side coupler 106.

In other words, when the bag 514 is being pulled or otherwise causing a pulling force F1 the decoupling force 702 may increase as the locking shelf 306 is biased towards the locking rim 202 as discussed herein compared to the required decoupling force 702 when the ostomate is uncoupling the bag side coupler 104 from the body side coupler 106. This increased decoupling force 702 allows the coupler assembly 100 to substantially prevent unintentional uncoupling while still providing an interface that is easy for the ostomate or other user to transition from the coupled configuration to the uncoupled configuration.

In another aspect of this disclosure, having the male portion 304 on the bag side coupler 104 may provide for coupling the bag side coupler 104 to the body side coupler 106 without binding of the bag side coupler 104 as it is pressed into the body side coupler 106. More specifically, users frequently couple the bag side coupler to the body side coupler of an ostomy appliance by first engaging a portion of the bag side coupler to the body side coupler. Then, the user often applies pressure around the perimeter of the bag side coupler to press the bag side coupler into the body side coupler as the user's fingers work around the perimeter. In the prior art designs, this coupling process causes the bag side coupler to bunch and bind as the user's fingers complete the perimeter of the bag side coupler. This makes coupling difficult and can result in the ostomy appliance being incompletely coupled by the user.

The present design places the male portion 304 on the bag side coupler 104 as discussed herein. In addition to all of the advantages discussed herein, this configuration allows the user to couple the bag side coupler 104 to the body side coupler 106 without the bag side coupler 104 binding as the user presses around the perimeter to coupler the bag side coupler 104 to the body side coupler 106. More specifically, the male portion 304 slides into the female receiver 206 as the user presses around the perimeter of the bag side coupler 104. This positions the male portion 304 of the bag side coupler 104 in alignment with the female receiver 206 as the user presses the bag side coupler 104 into the body side coupler 106 around the perimeter thereof. This alignment of the male portion 304 with the female receiver 206 prevents the bag side coupler 104 from bunching or binding during coupler. Accordingly, the teachings of the present disclosure avoids the binding and bunching issues of the prior art among other things.

Referring to FIG. 10, an alternative embodiment of a body side coupler 1000 is illustrated. The body side coupler 1000 may have substantially the same female receiver 206 as discussed herein, having a locking rim 202 and seal wall 208. However, instead of having an accordion membrane this body side coupler 1000 may have a convex flange 1002 molded with the female receiver 206. The convex flange 1002 may be offset from a planar surface 1004 of the body side coupler 1000.

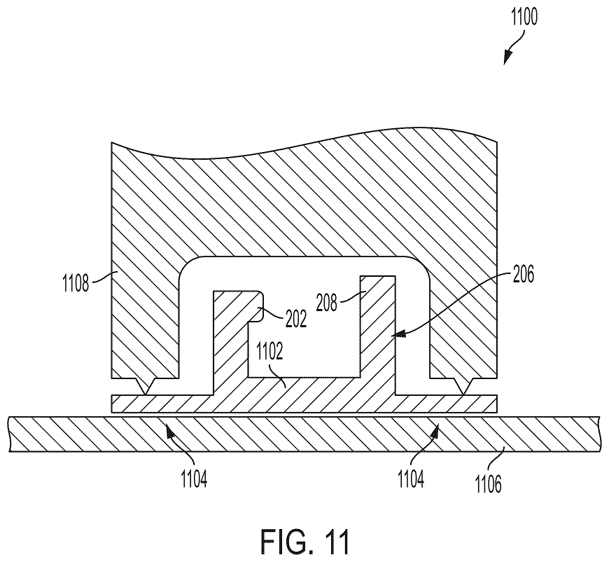

Referring now to FIG. 11, another embodiment of a body side coupler assembly 1100 is illustrated. In this embodiment, a body side coupler 1102 may have substantially the same female receiver 206 as discussed herein, having a locking rim 202 and seal wall 208 similar to other embodiments considered herein. However, instead of having an accordion membrane or convex flange, this coupler assembly 1100 may have coupler flanges 1104 extending from one or both sides of the female receiver 206. The coupler flanges 1104 may provide a location for the body side coupler 1102 to be coupled to an underlying wafer 1106 which may in turn have an adhesive to be coupled to an ostomate.

The coupler flanges 1104 may extend sufficiently away from the body side coupler 1102 to allow a welding assembly 1108 to be positioned along an outer surface of the coupler flanges 1104 to selectively weld the outer flanges 1104 to the underlying wafer 1106. The welding assembly 1108 may be an ultrasonic welding assembly that can be positioned along the coupler flanges 1104 to provide welds thereto to sufficiently couple the female receiver 206 to the wafer 1106.

While a welding assembly 1108 is discussed herein, the body side coupler 1102 may be coupled to the wafer 1106 with an adhesive or other known coupling method. Further, while coupler flanges 1104 are illustrated on either side of the female receiver 206, other embodiments contemplated herein may only have one of the two illustrated coupler flanges 1104. Further still, other embodiments considered herein may not utilize a coupler flange at all. In this embodiment, adhesive may be placed directly underneath the female receiver 206 to couple the body side coupler assembly 1100 to an ostomate.

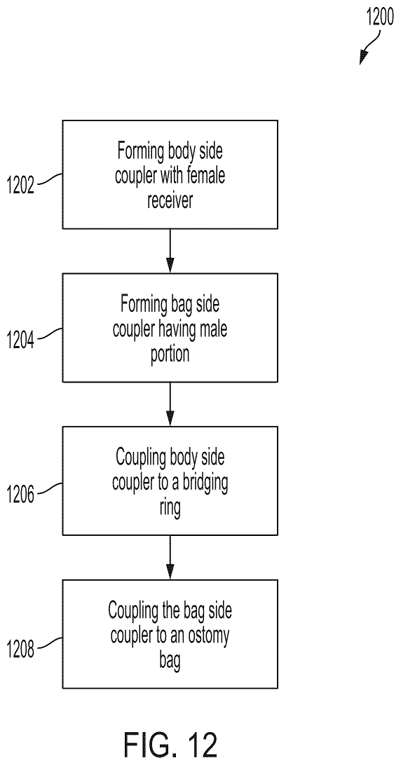

Referring now to FIG. 12, a method of manufacturing a coupler assembly for an ostomy appliance 1200 is illustrated. The method includes forming a body side coupler that defines a female receiver as outlined in box 1202. The body side coupler may be formed polyethylene or any other known material. Further, the body side coupler may be formed during an injection molding process or the like.

In box 1204 a bag side coupler is formed that defines a male portion sized to be selectively coupled to the female receiver. The bag side coupler may be formed of EVA or the like. Further, the bag side coupler may be formed during an injection molding process or other similar method.

In box 1206, the body side coupler is coupled to a bridging ring. As discussed herein, the body side coupler may be coupled to the bridging ring through ultrasonic welds, adhesives, or any other known coupling method.

Similarly, in box 1208, the bag side coupler is coupled to an ostomy bag. The bag side coupler may be coupled to the ostomy bag through ultrasonic welds, adhesives, or any other known coupling method.

In one embodiment of box 1202, an accordion membrane may be formed in the body side coupler between the female receiver and the bridging ring. Further in one contemplated embodiment of box 1202 the female receiver and the accordion membrane are formed together in the same manufacturing step. In one aspect of this embodiment, the coupling the body side coupler to the bridging ring from box 1206 comprises welding a portion of the accordion membrane to the bridging ring along a bridging ring weld.

Another aspect of this manufacturing method 1200 includes coupling an adhesive to the bridging ring to selectively couple the body side coupler to a user's abdominal wall. Yet another aspect of this manufacturing method 1200 includes welding the ostomy bag to a flange formed in the bag side coupler along a bag weld. The welding method may utilize ultrasonic welding or any other known welding technique.

Part of this manufacturing process 1200 may include forming a locking rim in the female receiver and forming a locking shelf in the male portion. The locking rim and locking shelf may be formed during the molding process or separately formed afterwards. Regardless, the locking rim and locking shelf are formed at locations that correspond with one another to substantially maintain the body side coupler and the bag side coupler in the coupled configuration. Similarly, a deflectable seal may be formed in the male portion during the molding process 1204 and a seal wall may be formed in the female receiver during the molding process 1202. The deflectable seal is formed at a location to provide a fluid seal between the bag side coupler and the body side coupler when in the coupled configuration. Further, the deflectable seal is formed at a location that allows the deflectable seal to provide a biasing force of the locking shelf towards the locking rim.

While this disclosure has been described with respect to at least one embodiment, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

1. A coupler assembly for an ostomy appliance, comprising:
a body side coupler defining a female receiver having a locking rim, the body side coupler configured to be selectively coupled to a user;
a bag side coupler defining a male portion having a locking shelf and sized to be selectively coupled to the female receiver, the bag side coupler configured to be coupled to an ostomy bag;
a deflectable seal defined about the male portion at a radially inward location from the coupler through-hole relative to the locking shelf; and
a seal wall defined in the female receiver wherein the deflectable seal contacts the seal wall to substantially seal an interior of the ostomy bag from the surrounding environment when the male portion is coupled to the female receiver;
wherein, when the male portion is coupled to the female receiver, providing a pulling force on the ostomy bag increases a decoupling force required to uncouple the male portion from the female receiver by biasing the locking rim and locking shelf towards one another.

2. The coupler assembly of claim 1, wherein the ostomy bag is coupled to the bag side coupler at a location radially outward of the locking shelf relative to a coupler through-hole.

3. The coupler assembly of claim 2, wherein the body side coupler is coupled to a bridging ring at a location radially inward from the locking rim relative to the coupler through-hole.

4. The coupler assembly of claim 1, comprising an accordion membrane defined in the body side coupler between the female receiver and a bridging ring weld.

5. The coupler assembly of claim 4, wherein the female receiver and the accordion membrane are formed of the same material in the same manufacturing process.

6. (canceled)

7. (canceled)

8. The coupler assembly of claim 1, further comprising a bridging ring coupled to the body side coupler on a first side and having an adhesive on a second side, the adhesive configured to be selectively coupled to a user's abdominal wall about a stoma.

9. The coupler assembly of claim 1, wherein the body side coupler is formed of polyethylene.

10. The coupler assembly of claim 9, wherein the bag side coupler is formed of EVA.

11. The coupler assembly of claim 1, wherein the female receiver of the body side coupler is welded directly to a wafer.

12. The coupler assembly of claim 1, comprising a flange defined by the bag side coupler extending radially outward relative to a coupler through-hole, the ostomy bag being welded to the bag side coupler along a surface of the flange.

13. The coupler assembly of claim 1, wherein the female receiver of the body side coupler has a convex flange that is offset from a planar surface of the female receiver.

14. A method of manufacturing a coupler assembly for an ostomy appliance, comprising:
forming a body side coupler that defines a female receiver having a locking rim;
forming a bag side coupler that defines a male portion having a locking shelf, the male portion sized to be selectively coupled to the female receiver;
forming a deflectable seal in the male portion at a radially inward location from a coupler through-hole relative to the locking shelf:
forming a seal wall in the female receiver wherein the deflectable seal contacts the seal wall to substantially seal an interior of the ostomy bag from the surrounding environment when the male portion is coupled to the female receiver;
coupling the body side coupler to a bridging ring;
coupling the bag side coupler to an ostomy bag;
wherein, the male portion is coupleable to the female receiver in a coupled configuration wherein providing a pulling force on the ostomy bag increases a decoupling force required to uncouple the male portion from the female receiver by biasing the locking rim towards the locking shelf.

15. The method of claim 14, further comprising forming an accordion membrane in the body side coupler between the female receiver and the bridging ring.

16. The method of claim 15, further comprising forming the female receiver and the accordion membrane in the same manufacturing step.

17. The method of claim 15, wherein the coupling the body side coupler to the bridging ring step comprises welding a portion of the accordion membrane to the bridging ring along a bridging ring weld.

18. The method of claim 14, further comprising coupling an adhesive to the bridging ring to selectively couple the body side coupler to a user's abdominal wall.

19. The method of claim 14, further comprising welding the ostomy bag to a flange formed in the bag side coupler along a bag weld.

20. The method of claim 14, wherein the locking rim and locking shelf correspond with one another to substantially maintain the body side coupler and the bag side coupler in the coupled configuration.

21. (canceled)

22. The method of claim 14, wherein the deflectable seal is positioned to provide a biasing force of the locking shelf towards the locking rim to maintain the coupled configuration.

23. The method of claim 14, wherein the body side coupler and bag side coupler are formed from an injection molding process.
